(19) United States
(12) Patent Application Publication
(10) Pub. No.: US 2021/0113850 A1
(43) Pub. Date: Apr. 22, 2021

(54) TERAHERTZ HEALTHY ROSARY STRUCTURE

(71) Applicant: HSIU-HUI SHEN, New Taipei City (TW)

(72) Inventor: HSIU-HUI SHEN, New Taipei City (TW)

(21) Appl. No.: 16/990,981

(22) Filed: Aug. 11, 2020

(30) Foreign Application Priority Data
Oct. 18, 2019 (TW) 108213766

(51) Int. Cl. A61N 5/00

(52) U.S. Cl. CPC A61N 5/00

(57) ABSTRACT

A terahertz healthy rosary structure includes a rope, a plurality of serial-connected rosary bead configured on the rope, a first rosary bead configured on the rope, and a plurality of terahertz layers formed on the surfaces of the serial-connected rosary beads and first rosary bead, where the two sides of the first rosary bead are respectively connected with the serial-connected rosary beads. When a user hangs the rope on the neck, uses it for singing Buddha, or places it close to the body, the terahertz layers will then emit terahertz waves. After the human body absorbs the terahertz waves, it can achieve the effects of promoting blood circulation, enhancing metabolism, and regulating autonomic nerves.

### TECHNICAL FIELD OF THE INVENTION

The present invention relates to a terahertz healthy rosary structure capable of promoting blood circulation, enhancing metabolism, and regulating autonomic nerves.

### DESCRIPTION OF THE PRIOR ART

Members of traditional religions use rosaries. However, these items do not have any substantial help in human bodies; they are used only for spiritual blessings; although they will not cause harm to human bodies, they will not have any benefits. If the rosaries can improve the physiological function, they will not only help the physical and mental health, but also help religious members to be more energetic when singing and chanting.

### SUMMARY OF THE INVENTION

The main object of the present invention is to provide a terahertz healthy rosary structure, allowing a human body to absorb terahertz waves emitted from terahertz layers, thereby achieving the effect of promoting blood circulation, enhancing metabolism, and regulating autonomic nerves.

The main structure of the present invention includes a rope, on which a first rosary bead and a plurality of serial-connected rosary beads are configured, and a terahertz layer is respectively covered on the surfaces of each of the serial-connected rosary beads and first rosary bead.

Whereby, when a user wants to use the present invention to chant sutras and sing Buddha, they only need to handhold the serial-connected rosary beads or first rosary bead on the rope and sing Buddha in a traditional way. At the same time, the terahertz layers formed on the serial-connected rosary beads and first rosary bead emit terahertz waves, and the human body will absorb the terahertz waves emitted form the serial-connected rosary beads and first rosary bead. when the body's terahertz wave amount is increased, the body temperature will increase, and the diseased cells rooted in the body will be repaired and corrected. In this way, it can increase the body's immunity, improve the body's metabolic system, regulate the autonomic nervous system, and make the mood feel happy. At the same time, it can also enhance the spirit of singing Buddha, so that religious members can concentrate on the ceremony and avoid distractions, sleepiness, and etc. occur when singing Buddha.

In addition, when the user does not need to chant sutras and sing Buddhist, the present invention can also be hung on the neck, worn on the hand, or placed near the human body, and the serial-connected rosary beads and first rosary bead on the rope will also continue emitting terahertz waves to improve the physiological functions of the human body according to the above structure and action. Since the terahertz layer emits terahertz wave any time, only carrying the present invention with you can continuously help the body and activate the cells in the body to achieve the effect of promoting blood circulation, enhancing metabolism, and regulating autonomic nerves.

### BRIEF DESCRIPTION OF THE DRAWINGS

FIG. 1 is a perspective view of a preferred embodiment of the present invention;

FIG. 2 is a cross-sectional view of the embodiment of the present invention;

FIG. 3 is a schematic view of the embodiment of the present invention when it used for singing Buddha;

FIG. 4 is a schematic view of the embodiment of the present invention when it is hung on a human body;

FIG. 5 is a schematic view of the embodiment of the present invention when it is worn on a human body;

FIG. 6 is a perspective view of another preferred embodiment of the present invention;

FIG. 7 is a perspective view of still another preferred embodiment of the present invention; and

FIG. 8 is a perspective view of yet another preferred embodiment of the present invention.

### DETAILED DESCRIPTION OF THE PREFERRED EMBODIMENTS

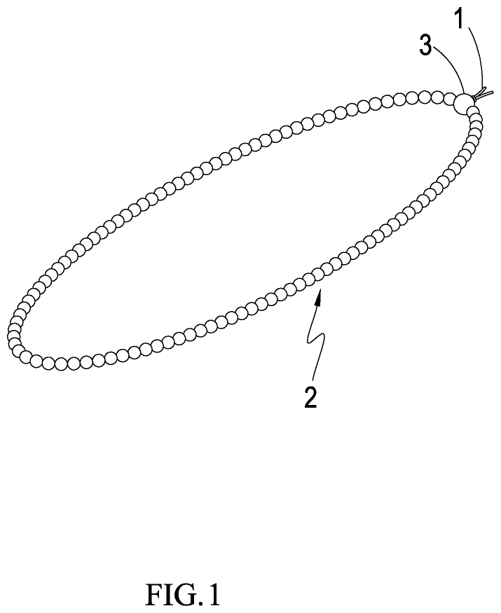

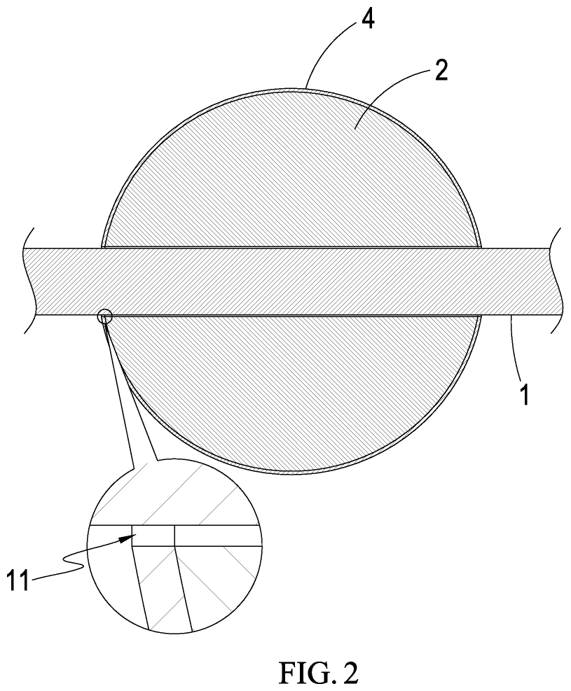

Referring to FIGS. 1 and 2, a terahertz healthy rosary structure of the present invention includes a rope 1, a plurality of serial-connected rosary beads 2, a first rosary bead 3 and a plurality of terahertz layers 4, where the plurality of serial-connected rosary beads 2 and the first rosary bead 3 are configured on the rope 1, and the terahertz layer 4 is respectively formed on the surface of each of the rosary beads 2 and the surface of the first rosary bead 3. In a preferred embodiment, for example, the serial-connected rosary beads 2 number 108, and each of the serial-connected rosary beads 2 and the first rosary bead 3 respectively have a through hole 11, allowing the rope 1 to be passed therethrough.

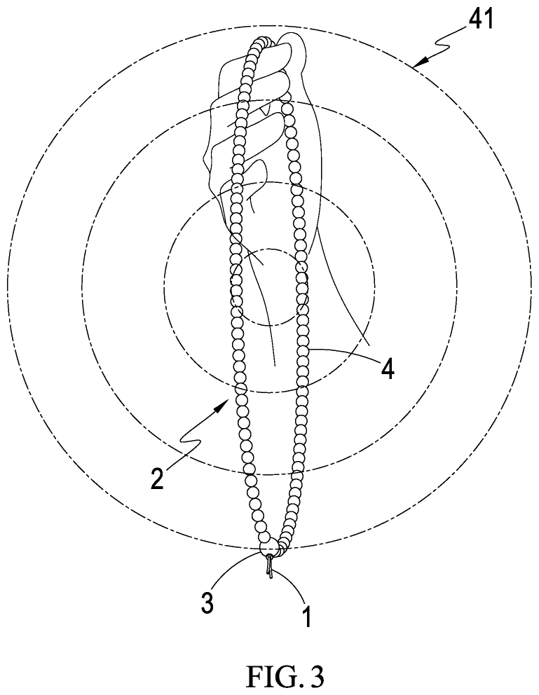

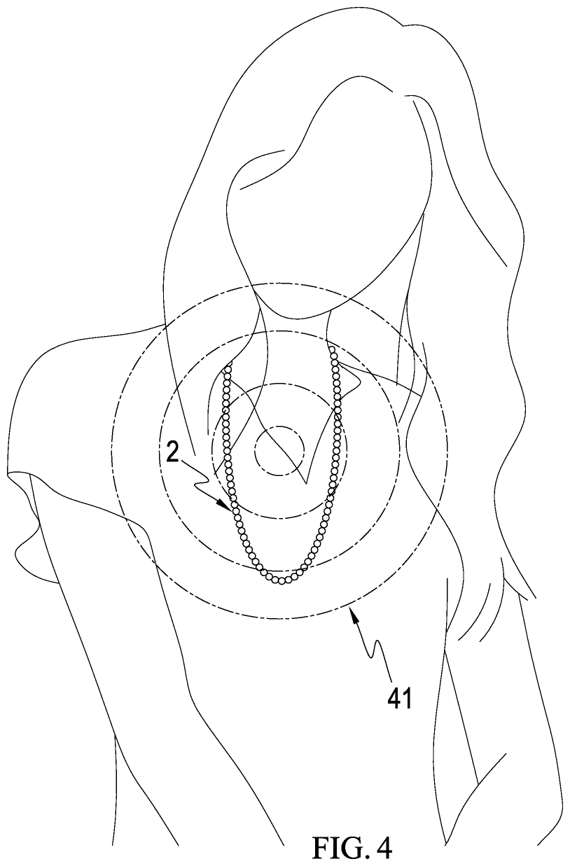

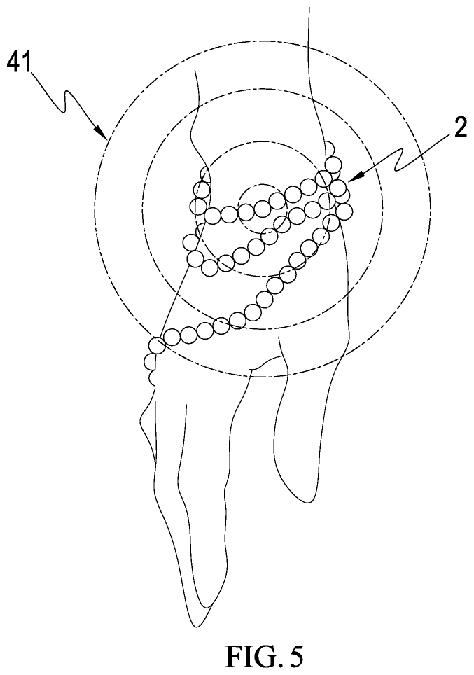

Referring to FIGS. 1 to 5, the terahertz layer 4 emits terahertz wave 41 all the time, and a human body only needs to be within the release range of the terahertz wave 41 to absorb it; religious members can absorb it when chanting and singing Buddha because all the rosary beads are not positioned away from the human body, such that it can absorb the terahertz waves 41 emitted from the terahertz layers 41 while chanting and singing Buddha.

The terahertz wave 41 is one of energy waves and refers to an electromagnetic wave having a frequency in the range of 0.1 THz to 10 THz and a wavelength between 0.03 mm and 3 mm, which also means that its wavelength is between the end of the microwave band and the start of the infrared band, such that most living creatures can release and absorb terahertz wave 41. Similarly, humans are no exception.

The terahertz wave 41 is the fluctuations in the origin of life and is also a quantum wave that is extremely beneficial to the human body. When a baby is born, the radiation amount of its terahertz wave 41 is surprisingly large, and with the increase of age, the emission of the terahertz wave 41 will gradually decrease. When a person dies at the end of his life, the radiation from their body drops to the microwatt level. From this, it can be seen that through the radiation of terahertz wave 41 in the human body, whether a person's vitality is strong or not can be judged.

Furthermore, when the terahertz wave 41 emitted by the human body comes into contact with the external terahertz wave 41, the resonance will be caused, allowing the water molecules inside the body to be activated and start to vibrate to make the volume of water clusters slowly shrink, thereby affecting the deep subcutaneous tissues of the human body, causing microvascular expansion, which in turn promotes blood circulation, enhances metabolism, and regulates autonomic nerves, etc. As a result, the body's immunity and self-healing ability will be improved.

The terahertz wave 41 has the following advantages besides the above-mentioned performances: activating molecules in the human body such as nucleic acid proteins, which is conducive to the restoration and balance of functions, and achieves the purpose of disease prevention and treatment; Improving the phagocytic function of cells in the organism, enhancing the cell performance of the human body, and benefiting the health of the human body; reducing the excitability of nerve endings, improving blood circulation, reducing edema, reducing the chemical and mechanical stimulation of nerve endings; effectively discharging toxins in the skin, slowing down spots, preventing common skin sub-health conditions such as acne.

When religious members are chanting and singing Buddha, they are prone to muscle soreness due to prolonged sitting or standing, but the muscle soreness can be relieved by improving the human body through the above terahertz wave 41. In addition, the religious members' spirits can also be kept up and their consciousnesses can also be restored when they feel tired and mentally weak. Whereby, the ritual of chanting and singing Buddha is no longer just for salvation and comfort in the soul, but also to substantially change the functions of the body, so that the body, mind, and soul are sublimated.

Furthermore, if there is no need to chant sutras and chant Buddha, a user may also wear the rope 1 on their body, for example, hang on their hand or place on the position close to their body; the terahertz layers 4 on the first rosary bead 3 and serial-connected rosary beads 2 will also release terahertz waves 41 to improve human body's functional system; As long as the distance between the beads and human body is within the radiation range of terahertz wave 41, it can achieve the effects of promoting blood circulation, enhancing metabolism, and regulating autonomic nerves.

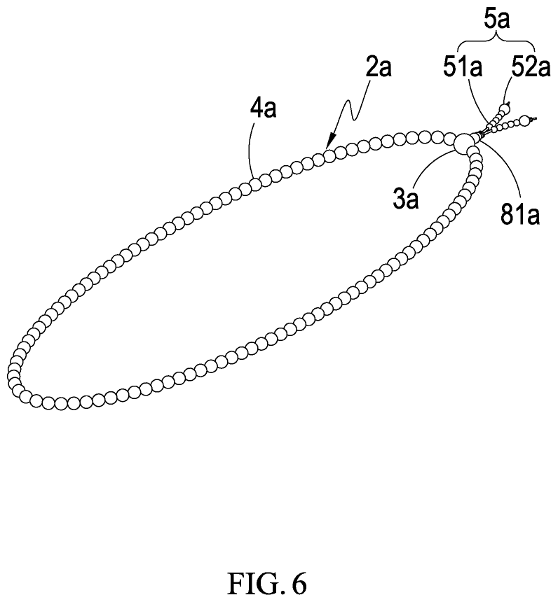

Referring to FIG. 6, one side of the first rosary bead 3a, in a preferred embodiment, is further configured with at least one first extended bead assembly 5a; a first connection rosary bead 81a is connected between the first rosary bead 3a and first extended bead assembly 5a, and the terahertz layer 4a is formed on the surface of the first extended bead assembly 5a. In the embodiment, each of the first extended bead assemblies 5a has a plurality of first extended rosary beads 51a and a first hanging rosary bead 52a. As an example, the first extended bead assemblies 5a number 2, the first extended rosary beads 51a 10, and serial-connected rosary beads 2a 108 the same as the above embodiment.

With the above structures, the present invention can then be allowed to produce different forms of rosary structures, thereby conforming to different users' requirements, but the present embodiment only list a different implementation pattern, and its size and arrangement are not limited.

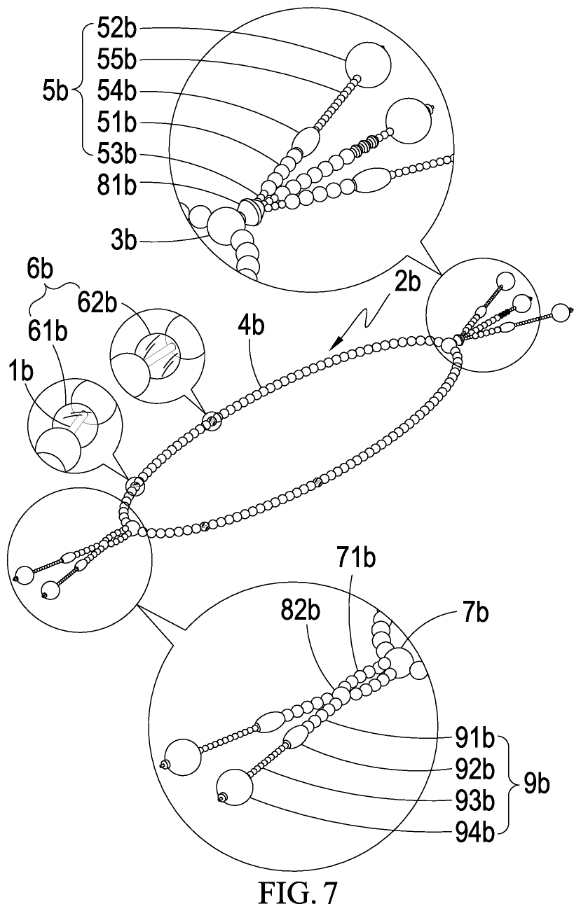

Referring to FIG. 7, a plurality of segmenting rosary beads 6b and a second rosary bead 7b, in a preferred embodiment, are further configured on the rope 1b, where the two sides of the segmenting rosary bead 6b are respectively connected with the serial-connected rosary bead 2b, and the two sides of the second rosary bead 7b are also similarly connected with the serial-connected rosary bead 2b. The segmenting rosary bead 6b is a transparent bead as an example, but the present invention is not so limited. One side of the first rosary bead 3b is connected with at least one first connection rosary bead 81b, and one side of the first connection rosary bead 81b away from the first rosary bead 3b is connected with at least one first extended bead assembly 5b, where the first connection rosary bead 81b and first extended bead assembly 5b are configured on the rope 1b. Furthermore, each of the first extended bead assemblies 5b has a plurality of balls 53b, a plurality of first extended rosary beads 51b, a first join rosary bead 54b, a plurality of second balls 55b and a first hanging rosary bead 52b, where one side of the first connection rosary bead 81b is connected with the first balls 53b, one side of the first balls 53b the first extended rosary beads 51b, one side of the first extended rosary beads 51b the first join rosary bead 54b, one side of the first join rosary bead 54b the second balls 55b, and one side of the second balls 55b the first hanging rosary bead 52b.

One side of the second rosary bead 7b is connected with a plurality of second extended rosary beads 71b, one side of the second extended rosary beads 71b a second connection rosary bead 82, one side of the second connection rosary bead 82b at least one second extended bead assembly 9b, and the second extended rosary beads 71b, second connection rosary bead 82b and second extended bead assemblies 9b are configured on the rope 1b. Furthermore, each of the second extended rosary bead assemblies 9b has a plurality of third extended rosary beads 91b, a second join rosary bead 92b, a plurality of third balls 93b and a second hanging rosary bead 94b, one side of the second connection rosary bead 82b is connected with the third extended rosary beads 91b, one side of the third extended rosary beads 91b the second joint rosary bead 92b, one side of the second join rosary bead 92b the third balls 93b, and one side of the third balls 93b the second hanging rosary bead 94b.

The terahertz layer 4b is respectively configured on the surfaces of the first rosary bead 3b, second rosary bead 7b, serial-connected rosary beads 2b, first extended rosary beads 1b, second extended rosary beads 71b, third extended rosary beads 91b, first join rosary bead 54b, second join rosary bead 92b, first balls 53b, second balls 55b, third balls 93b, first hanging rosary bead 53b, and second hanging rosary bead 94b.

The size of the first hanging rosary bead 52b and second hanging rosary bead 94b are the same, the sizes of the first rosary bead 3b and second rosary bead 7b are the same, the sizes of the first extended rosary bead 51b, second extended rosary bead 71b and third extended rosary bead 91b are the same, The size of the first ball 53b, second ball 55b and third ball 93b are equal, the size of the first hanging rosary bead 52b is larger than the one of the first rosary bead 3b, the size of the first rosary bead 3b is larger than the one of the serial-connected rosary bead 2b, the size of the serial-connected rosary bead 2b is larger than the one of the first extended rosary bead 51b, and the size of the first extended rosary bead 51b is larger than the one of the first ball 53b.

As an example, the first extended bead assemblies 5h number 3, the second extended bead assemblies 9b2, and the serial-connected rosary beads 2b 108; the segmenting rosary beads 6b includes a plurality of first segmenting rosary bead 61b and a plurality of second segmenting rosary bead 62b, and the first segmenting rosary beads 61b and second segmenting rosary beads 62b respectively number 2; the serial-connected rosary beads 2b between the second rosary bead 7b and first segmenting rosary bead 61b number 7, the serial-connected rosary beads 2b between the first segmenting rosary bead 61b and second segmenting rosary bead 62b 14, and the serial-connected rosary beads 2b between the second segmenting rosary bead 62 and first rosary bead 3b 33.

The structure of the present embodiment is a rosary structure used by Soka Gakkai International (SGI), and SGI members are many. Therefore, the number of people who use this kind of rosary structure to chant sutras and sing Buddha is countless. With the structure of the above terahertz layer 4b, the members of SGI or users of this kind of rosary can obtain the effects of promoting blood circulation, enhancing metabolism, and regulating the autonomic nerves when using the rosary, thereby benefiting more religious members in the world.

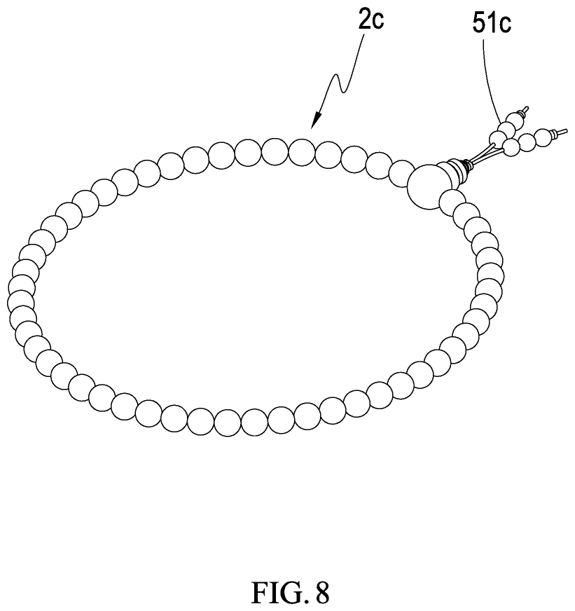

Referring to FIG. 8, the number of serial-connected rosary beads 2c is changed to 54, and the number of the first extended rosary beads 51c is changed to 6. This embodiment only shows the implementation state of serial-connected beads 2c with a different number, so as to show that the number is not limited.

I claim:

1. A terahertz healthy rosary structure, comprising:
a rope;
a plurality of serial-connected rosary beads, each thereof configured on said rope;
a first rosary bead, configured on said rope, and two sides of said first rosary bead respectively connected with each of said serial-connected rosary beads; and
a plurality of terahertz layer, each thereof formed on surfaces of each of said serial-connected rosary beads and said first rosary bead.

2. The structure according to claim 1, wherein said rope has a plurality of segmenting rosary beads, and two sides of each of said segmenting rosary bead are respectively connected with each of said serial-connected rosary beads.

3. The structure according to claim 2, wherein said serial-connected rosary beads number 108, and said segmenting rosary beads 4.

4. The structure according to claim 1, wherein one side of said first rosary bead has at least one first extended bead assembly, said first extended bead assembly is configured on said rope, and said terahertz layers are formed on a surface of said first extended bead assembly.

5. The structure according to claim 4, wherein a first connection rosary bead is connected between said first rosary bead and first extended bead assembly, and said first connection rosary bead is configured on said rope.

6. The structure according to claim 4, wherein said first extended bead assembly has a plurality of first balls, a plurality of first extended rosary beads, a first join rosary bead, a plurality of second balls and a first hanging rosary bead, each of said first balls, each of said first extended rosary bead, said first join rosary bead, each of said second balls and said first hanging rosary bead are connected to each other in sequence, a size of said first hanging rosary bead is larger than the one of said first rosary bead, a size of said first rosary bead the one of each of said serial-connected rosary bead, a size of each of said serial-connected rosary bead the one of said first extended rosary bead, a size of each of said first extended rosary bead the ones of each of said first balls and each of said second balls, and said first extended bead assemblies number 3.

7. The structure according to claim 1, wherein said rope has a second rosary bead, two sides of said second rosary bead are respectively connected with each of said serial-connected rosary beads, and said terahertz layer is formed on said second rosary bead.

8. The structure according to claim 7, wherein one side of said second rosary bead is connected with a plurality of second extended rosary beads, said second extended rosary beads are in connection with a second connection rosary bead, said terahertz layers are formed on surfaces of said second extended rosary beads, and said second extended rosary beads and said second connection rosary bead are configured on said rope.

9. The structure according to claim 8, wherein one side of said second connection rosary bead is connected with at least one second extended bead assembly, said second extended bead assembly is configured on said rope, and said terahertz layers are formed on said second extended bead assembly.

10. The structure according to claim 8, wherein said second extended bead assembly has a plurality of third extended rosary beads, a second join rosary bead, a plurality of third balls and a second hanging rosary bead, each of said third extended rosary beads, said second join rosary bead, each of said third balls and said second hanging rosary bead are connected to each other in sequence, a size of said second hanging rosary bead is larger than the one of said first rosary bead, a size of said first rosary bead is equal to the one of said second rosary bead, a size of said second rosary bead is larger than the one of each of said serial-connected rosary beads, a size of each of said serial-connected rosary beads is larger than the one of each of said third extended rosary beads, a size of said third extended rosary beads is larger than the one of each of said third balls, and said second extended bead assemblies number 2.
